(19) United States
(12) Patent Application Publication
(10) Pub. No.: US 2024/0307634 A1
(43) Pub. Date: Sep. 19, 2024

(54) ELECTRICAL CONDUCTIVITY SENSOR SYSTEM AND DEVICE WITH REAL-TIME FEEDBACK

(71) Applicant: Acies Medical LLC, Saint Paul, MN (US)

(72) Inventor: Brian L. Norling, Santa Barbara, CA (US)

(73) Assignee: Acies Medical LLC, Saint Paul, MN (US)

(21) Appl. No.: 18/559,879

(22) PCT Filed: May 23, 2022
(86) PCT No.: PCT/US2022/030596

§ 371 Date: Nov. 9, 2023

Related U.S. Application Data
(60) Provisional application No. 63/191,723, filed on May 21, 2021.

(51) Int. Cl. A61M 5/42

(52) U.S. Cl. CPC A61M 5/427; A61M 2205/3306; A61M 2230/65

(57) ABSTRACT

A real-time impedance measuring needle system having a hollow needle for administering medical fluids having a distal end, and a proximal end, which includes an opening configured to receive medical fluids through a fluid channel. The system may also have at least two conductors located on the distal end of the needle near the opening configured to deliver medical fluids, and at least one detector configured to sense electrical current running between the at least two conductors through a biological tissue.

### CROSS-REFERENCE TO RELATED APPLICATIONS

This application claims the benefit of U.S. provisional Application No. 63/191,723, filed May 21, 2021 and titled REAL-TIME FEEDBACK OF RESISTANCE/IMPEDENCE.

The aforementioned patent applications are hereby incorporated by reference, such incorporation being limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

### FIELD OF THE DISCLOSURE

The present disclosure relates to a needle with sensors that is useful for real-time feedback in medical procedures on various tissues.

### BACKGROUND

Efforts to improve surgical outcomes and cost structure, particularly with spinal surgery or dental procedures, have led to increased use of minimally invasive procedures. These procedures often use image-guided modalities such as fluoroscopy, CT, nerve stimulators, and, more recently, the Doppler ultrasound test. While often involving less risk than surgery, minimally invasive spinal procedures, pain management procedures, nerve blocks, ultrasound guided interventions, biopsy, and percutaneous placement or open intra-operative placement continue to carry risks of ineffective outcome and iatrogenic injuries, such as infection, stroke, paralysis and death due to penetration of various structures including, but not limited to, organs, soft tissues, vascular structures, and neural tissue such as, catastrophically, the spinal cord. Injuries can occur regardless of practitioner experience because a surgical instrument must proceed through several layers of bodily tissues and fluids to reach the desired space in the spinal canal.

To illustrate, the intrathecal (or subarachnoid) space of the spinal region, where many medications are administered, houses nerve roots and cerebrospinal fluid (CSF) and lays between two of the three membranes that envelope the central nervous system. The outermost membrane of the central nervous system is the dura mater, the second is the arachnoid mater, and the third, and innermost membrane, is the pia mater. The intrathecal space is in between the arachnoid mater and the pia mater. To get to this area, a surgical instrument may need to first get through skin layers, fat layers, the interspinal ligament, the ligamentum flavum, the epidural space, the dura mater, the subdural space, and the intrathecal space. Additionally, in the case of a needle used to administer medication, the entire needle opening must be within the sub-arachnoid space.

Because of the complexities involved in inserting a surgical instrument into the intrathecal space, penetration of the spinal cord and neural tissue is a known complication of minimally invasive spine procedures and spine surgery. Additionally, some procedures require the use of larger surgical instruments. For example, spinal cord stimulation, a form of minimally invasive spinal procedure wherein small wire leads can be inserted in the spinal epidural space, may require that a 14-gauge needle be introduced into the epidural space in order to thread the stimulator lead. Needles of this gauge are technically more difficult to control, posing a higher risk of morbidity. Complications can include dural tear, spinal fluid leak, epidural vein rupture with subsequent hematoma, and direct penetration of the spinal cord or nerves with resultant paralysis. These and other high-risk situations, such as spinal interventions and radiofrequency ablation, can occur when a practitioner is unable to detect placement of the needle or surgical apparatus tip in critical anatomic structures.

At present, detection of such structures is operator dependent, wherein operators utilize tactile feel, contrast agents, anatomical landmark palpation and visualization under image-guided modalities. The safety of patients can rely upon the training and experience of the practitioner in tactile feel and interpretation of the imagery. Even though additional training and experience may help a practitioner, iatrogenic injury can occur independently of practitioner experience and skill because of anatomic variability, which can arise naturally or from repeat procedures in the form of scar tissue. Fellowship training in some procedures, such as radiofrequency ablation, may not be sufficiently rigorous to ensure competence; even with training, outcomes from the procedure can vary considerably. In the case of epidural injections and spinal surgery, variability in the thickness of the ligamentum flavum, width of the epidural space, dural ectasia, epidural lipomatosis, dural septum, and scar tissue all can add challenges to traditional verification methods even for highly experienced operators. Additionally, repeat radiofrequency procedures done when nerves regenerate, often a year or more later, are often less effective and more difficult because the nerves' distribution after regeneration creates additional anatomic variability.

### SUMMARY OF THE INVENTION

An electrical conductivity sensor system is disclosed that provides real-time feedback to a medical practitioner regarding the type of tissue that the tip of a hollow needle is in contact with before injection of a medical fluid. This sensor system can be useful for medical procedures where the precise location of the placement of the medical fluid is desired.

An electrical resistance sensor is disclosed that is useful for real-time tissue identification. The sensor includes a hollow hypodermic needle capable of injecting medical fluids into living tissue. An electrical resistivity probe is at least partially encased within the hollow hypodermic needle. The disclosed sensor also includes a conductivity tip at one end of the electrical resistivity probe. The disclosed sensor is useful for identifying tissue in real-time during a medical procedure. The disclosed sensor can further include a syringe for delivering medical fluids to the hollow hypodermic needle. In some embodiments, a sensor can include two or four electrical resistivity probes all partially encased in the needle.

This disclosure may be useful for measuring over 21 different types of tissue or biological material that have different impedances. Using the unique natural structure of the biological material, the sensors and conductors may be arranged in an advantageous way to allow for impedance measurement across biological tissue to help identify potential needle injection site. The system may further have electrical components for measuring resistance and determining voltage, and send an output for a clinician to read levels or measured results of impendence to determine if the needle is in the correct location prior to delivering fluid or medicine to the patient.

In another aspect, a method for retracting a fiber optic sensor is disclosed that includes providing a fiber optic sensor retraction system according to the disclosure above. The method further includes inserting the needle containing the fiber optic wavelength sensor into a patient and using the fiber optic wavelength sensor to properly place the needle. The method includes retracting the fiber optic wavelength sensor and then administering medical fluids through the hollow needle.

### BRIEF DESCRIPTION OF THE DRAWINGS

FIG. 1 is an illustrative example of an embodiment of a real-time sensor system according to the present disclosure.

FIG. 2 is an illustrative example of an embodiment of a real-time sensor system according to the present disclosure.

FIG. 3 is an illustrative example of a cross sectional view of an embodiment of a real-time sensor system according to the present disclosure.

FIG. 4 is an illustrative example of an embodiment of the tip of a real-time sensor system according to the present disclosure.

FIG. 5 is an illustrative example of an embodiment of the tip of a real-time sensor system according to the present disclosure.

FIG. 6 is an illustrative example of an embodiment of a real-time sensor system according to the present disclosure.

FIG. 7 is an illustrative example of an embodiment of a real-time sensor system according to the present disclosure.

FIG. 8 is an illustrative example of an embodiment of a real-time sensor system according to the present disclosure.

FIG. 9 is an illustrative example of an embodiment of a real-time sensor system according to the present disclosure.

FIG. 10 is an illustrative example of an embodiment of a real-time sensor system according to the present disclosure.

FIG. 11 is an illustrative example of an embodiment of a real-time sensor system according to the present disclosure.

FIG. 12 is an illustrative example of an embodiment of a real-time sensor system according to the present disclosure.

FIG. 13 is an illustrative example of an embodiment of a real-time sensor system according to the present disclosure.

FIG. 14 is an illustrative example of a connection between the fiber and processing components according to the present disclosure.

FIG. 15 is an illustrative example of a circuit for measuring resistance according to the present disclosure.

FIG. 16 is an illustrative example of a circuit for measuring resistance with multiple resistors, according to the present disclosure.

### DETAILED DESCRIPTION

The present disclosure relates to a real-time impedance measuring needle system used to detect biological substances, such as bodily fluids and tissues, including blood. Various embodiments of real-time impedance measuring needle systems are described in detail with reference to the drawings, wherein like reference numerals may represent like parts and assemblies throughout the several views. Reference to various embodiments does not limit the scope of the real-time impedance measuring needle system disclosed herein. Additionally, any examples set forth in this specification are not intended to be limiting and merely set forth some of the many possible embodiments for the real-time impedance measuring needle system. It is understood that various omissions and substitutions of equivalents are contemplated as circumstances may suggest or render expedient, but these are intended to cover applications or embodiments without departing from the spirit or scope of the disclosure. Also, it is to be understood that the phraseology and terminology used herein are for the purpose of description and should not be regarded as limiting.

Fiber optic waveguides can be placed in the interior of hollow needles useful in medical procedures. The proper placement of the needles can be critical in the success of medical procedures such as surgical procedures on the nervous system (i.e. spinal surgery) or dental procedures. The fiber optic waveguide can be used as part of a sensor system that can detect important biological structures. For example, when trying to inject an anesthetic into a patient in a dental setting, it can be desirable to avoid injecting the medication into a blood vessel, which can cause negative undesirable systemic reactions in a patient. A fiber optic sensor in the injection needle can be a part of a detection system that detects iron ions (in blood, for example) and can indicate the improper placement of the needle. In this case the needle can be relocated so that the anesthetic is properly injected. In some embodiments, the sensor may be located level with the needle tip or bevel. This location may allow the sensor to detect blood or other biomarkers at or near the tip of the needle, which is where the medicine or fluid would be delivered.

In another example, spinal surgeons may want to locate various biological structures when placing a needle for administration of a medicament. In these cases, the surgeon may wish to find spinal fluid or avoid other physiological structures. A fiber optic detector can be placed into the needle which can be part of a detection system that can identify biological tissues in contact with the needle opening.

In normal operation, the surface of the fiber optic waveguide sensor within a hollow needle can be roughly coplanar with the tip of the needle (such as, a Quincke needle). The fiber optic waveguide can be much smaller than the needle bore, allowing for injection of fluids, such as medicaments, to flow past the fiber optic waveguide. In some modalities, the entire needle bore can be filled with the fiber optic waveguide. In these cases, the needle may retract or remove the fiber optic waveguide prior to the injection of fluids.

The removal of fiber optic waveguides can be done by hand by pulling the optical fiber waveguide into a Y-junction of guided syringe placement and delivery system until the fiber optic waveguide clears the needle bore. Hand removal of the optical fiber waveguide can be a significant operation by the surgeon or surgical staff at the precise point of injection, when movement of the needle tip is undesirable. Such a manual procedure also can require extra time for the whole procedure. Additionally, if a hard stop for fiber retraction is not included in the delivery system, it is possible for a leak to be created. However, this is unlikely based upon the approach of using a flexible polymer membrane similar to that used on a sealed medicine bottle or vial.

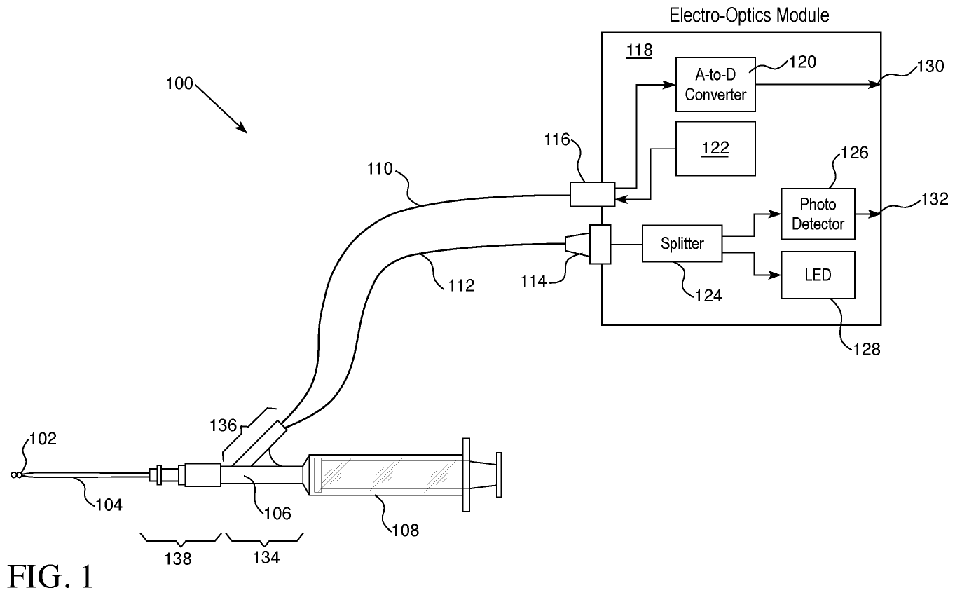

FIG. 1 is an illustration of one embodiment of a real-time impedance measuring needle system 100 (hereinafter referred to as the “system”). At the distal end of a hollow needle 104 (e.g., the end opposite the syringe), the tip of hollow needle 104 may include or attach to at least two conductors 102. These conductors 102 may be connected through a connection point such as a flange (not illustrated) that may maintain an electrical connection with the at least two conductors 102. When the needle tip is placed in a target location on the patient, the at least two conductors 102 can be in contact with biological tissue of the patient. A current may then run between the at least two conductors 102. The sensed signal may then be transmitted further in the system. Hollow needle 104 may connect with a Y-Coupler 106, which may branch in two different directions away from hollow needle 104. A first branch, or arm 136, may communicate an electrical signal via an electrical cable 110. First branch, or arm 136, may also communicate light signals optically via an optical fiber cable 112. Hollow needle 136 may connect to a third branch, or arm 138, of the Y-Coupler for delivering fluids to the biological tissue.

In first arm 136, electrical cable 110 may connect with a computing device (not illustrated), such as an electro-optics module or device 118. The connection may be wired or wireless between the Y-Coupler 106 and electro-optics device 118 through an electrical connector 116. Electrical connector 116 may connect to electro-optics device 118, which may include a signal converter 120 (such as an analog to digital or digital to analog) and, in some embodiments, a current source 122. After signal converter 120, the signal may be sent to a resistance output 130. The output at resistance output 130 may be displayed or communicated as a number on an output display, a light, a sound, a notification, a hepatic, and combinations thereof, wherein each can indicate or be based on the measured resistance of the biological tissue. There are many different embodiments that the output can be communicated as useful for the clinician.

In one embodiment, first arm 136 of the Y-Coupler 106 may also connect to an optical fiber cable 112, which can carry information based on iron ion content gathered from the sensors on the tip of the optical fiber at hollow needle 104 tip. Optical fiber cable 112 can send the information to electro-optics device 118. Optical fiber cable 112 can connect to electro-optics device 118 through fiber connector 114. Electro-optics device 118 may have a splitter 124 that receives the information via fiber connector 114. The biomarker information, such as iron ion content, may then be split by splitter 124 with a first signal that may be sent to a photo detector 126 and the other, second signal that may be sent to LED 128. The photo detector 126 may then send biomarker information to an output display 132. The biomarker content output may be displayed or communicated in a number of ways, such as a number displayed on an output display 132, a light, a sound, a notification, a hepatic, and combinations thereof, wherein each can indicate or be based on the measured resistance of the biological tissue. There are many different embodiments that the output can be communicated as useful for the clinician.

When the output indicates that the user is in the wrong tissue or detects certain biological material or tissue that is not desired for delivery of the medical fluid, then the user may reposition the needle to a different location for further determination of a location for medical fluid delivery. When the user determines that the needle location is suitable for delivery, the user may deliver the medical fluid into the tissue that the needle has entered. In some embodiments, the delivery may be automated or computing device controlled when the delivery site is suitable for the medical fluid injection.

As described herein, the electro-optics device may have more than one embodiment. The two connections to the first arm are only one embodiment of many that can transfer the at least two types of information.

Y-Coupler 106 can have a second branch that splits off where first arm 136 begins and second branch or arm 134 connects to a syringe 108. The connection of second arm 134 may create a continuous fluid channel (not illustrated) between syringe 108 and hollow needle 104 for delivery of medical fluids in syringe 108. There are several embodiments of hollow needles that may be used in coordination with the Y-Coupler 106, syringe 108, and communication system (i.e., electrical cable 110, optical fiber cable 112, and other components connected thereto).

In the simplest embodiment disclosed herein, a spring can be employed with a trigger release to rapidly extract the fiber. This embodiment is illustrated in FIG. 1 and may incorporate a spring for stored motive force. As shown, a trigger for release of the spring is provided as well as a polymer seal system to maintain sterile integrity and pressure in the system. When the trigger is engaged, the fiber optic waveguide (which can host the electrical cable 110, the optical fiber cable 112, or both) can be instantly extracted from the needle with minimal motion of the needle tip.

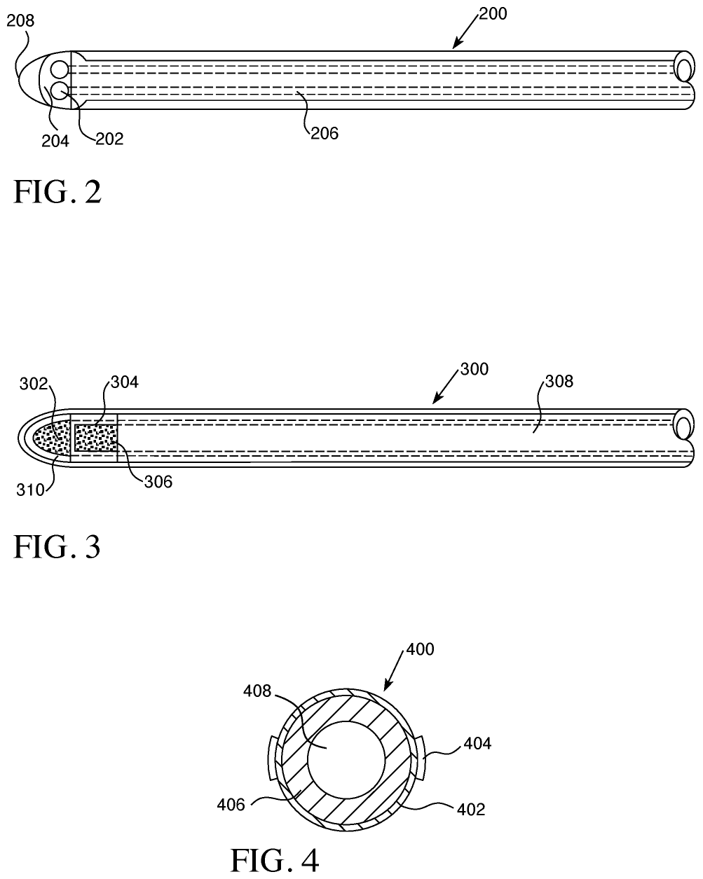

More specifically, a first embodiment of a hollow needle, such as hollow needle 200, is illustrated in FIG. 2. The wall of hollow needle 200 may have layers. In an interior of hollow needle 200, conductors 202 can be attached or integrally connected to the inner wall. Conductors 202 may run along hollow needle 200. A layer of outer insulation 206 may be between conductors 202 and the inner wall of hollow needle 200 to help insulate the electrical signal. In some embodiments, and as illustrated in FIG. 2, the outer insulation 206 may be a coating or layer over the conductors 202 that extends the length of conductors 202. In other embodiments, the outer insulation 206 may be a layer or lining of the needle to prevent the electrical signal from transferring to the hollow needle 200 and maintaining the electrical signal along conductors 202, so the outer insulation 206 may be at least where the conductors are and between other metallic or conductive materials, such as hollow needle 200. The outer insulation 206 may be outer side relative to the conductors (i.e., wrapped around the conductors) but inside of hollow needle 200, lining the inner wall of hollow needle 200 at least where the conducts 202 are located. Such an insulation material may be made of polyimide or similar material, but other materials are possible for electrical signal insulation. In the illustration of FIG. 2, two conductors are present, but more can be added to the system. In the embodiment illustrated, at least two conductors are located at a needle tip 208 of hollow needle 200, located at a distal end of the needle.

Optical fiber (not illustrated) may be a waveguide, which may moveably or slidably fit within the lumen of fluid channel and may further have an optical coupler. In some embodiments, optical fibers may have an insulting layer, such as inner insulating layer 204, that runs along the conduction paths, so that the electrical signal is not impacted by any optical fibers that may be placed inside hollow needle 200. In some embodiments, inner insulating layer may be on the inner side of conductors 202 and in other embodiments, inner insulating layer 204 may be part of an optical fiber outer coating or layer (not illustrated). In other embodiments, the at least two conductors may have inner insulating layer 204 on the conductors.

FIG. 3 is a similar view as FIG. 2 but is further detail of a hollow needle 300 with insulation layers. The layers may be from the inner wall of a hollow needle 300 and be layered to the outside layer, so that inner means closer to an inner wall and outer means closer to an outer wall. In one embodiment, an inner conductor 310 may be present that senses or collects the electric signal, as described herein. An intermediate insulation 304 may be between inner conductor 310 and outer conductor 306. In some embodiments, there may be an inner insulation 302 that is out the inner side of inner conductor 310 (not illustrated) or on the outer edge of an optical fiber (not illustrated). In some embodiments, as illustrated in FIG. 3, there may be an outer insulation layer 308 that is the outer most layer relative to the other components within needle 300.

FIG. 4 is a cross-sectional view of a hollow needle 400, as described herein. FIG. 4 shows a conductor 404 on the outer side of an insulation 402. Insulation 402 may coat an optical fiber 406 with a core 408. In some embodiments, optical fiber 406 (with core 408) may be not be present in hollow needle 400. In other embodiments, optical fiber 406 may have a coating on the outer edge, such as insulation 402. Thus, insulation 402 may be a layer or coating on optical fiber 406, or insulation 402 may coat the inner wall of hollow needle 400. In some embodiments, FIG. 4 may be a cross sectional view of FIGS. 10 and 11 with two conductors on opposing sides on hollow needle 400.

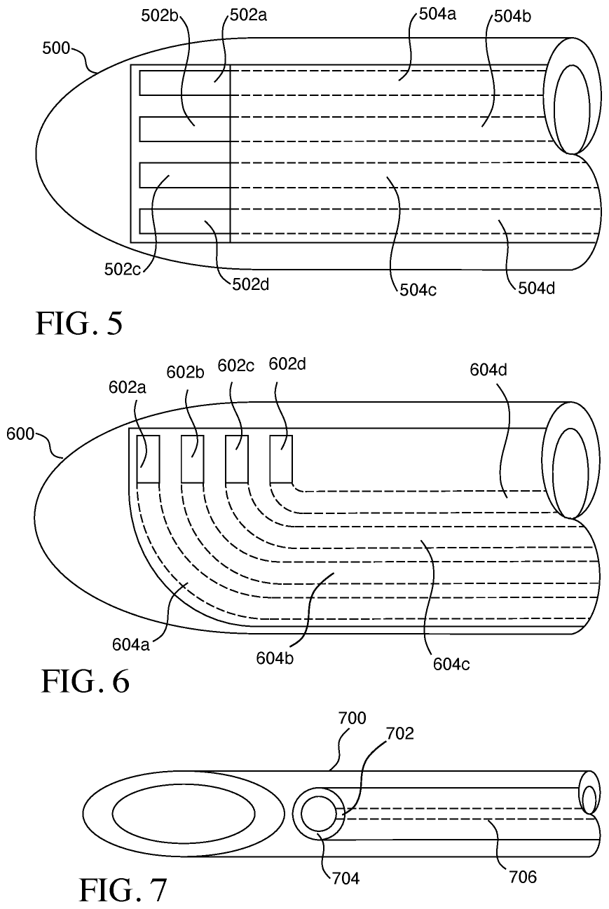

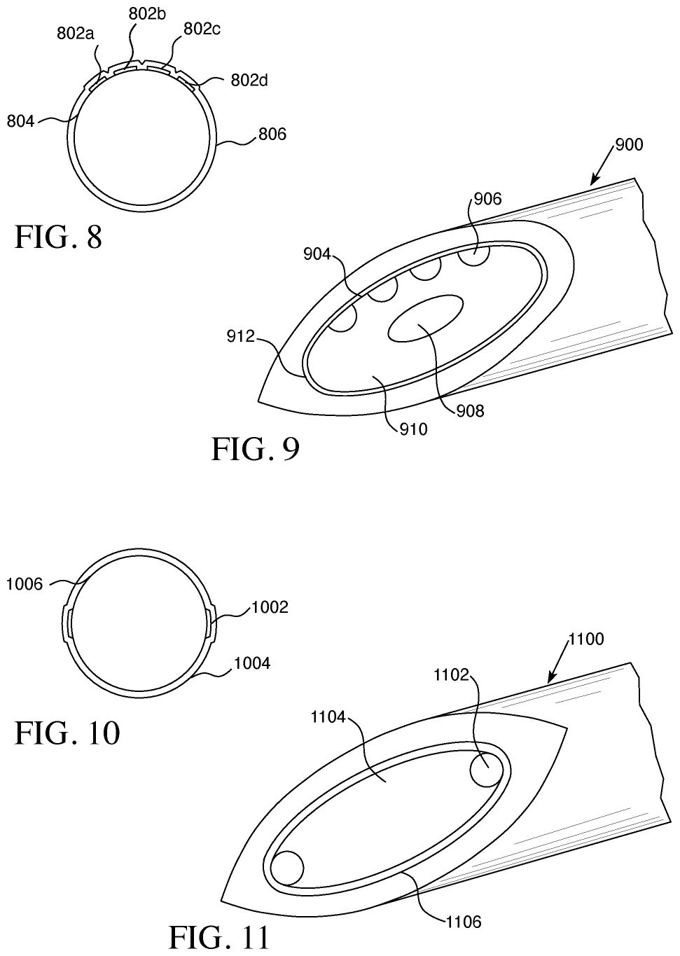

FIG. 5 illustrates a second embodiment of a hollow needle, wherein a tip of a hollow needle 500 has more than two conductors. In FIG. 5, there are four conductor 502a-d (collectively). Each of conductors 502a-d may extend or connect to a respective conducting path 504a-d that runs along hollow needle 500. There may be a respective conducting path 504 to connect with each respective conductor 502, such as conductor 502a connecting to conducting path 504a, 502b connecting to conducting path 504b, 502c connecting to conducting path 504c, and 502d connecting to conducting path 504d. In the embodiment of FIG. 8, metallization, electrodes, or conductions pads (802 of FIG. 8) are located on an inner wall. The embodiment of FIG. 5 illustrates conduction paths extending from the conduction pads respectively along hollow needle 500. The paths carry electrical signals to a computing device for signal analysis and resistance determination (see, e.g., FIG. 1). FIG. 5 is a cutout of hollow needle 500 (for the system, see FIG. 1 illustrating hollow needle 104).

FIG. 6 illustrates a third embodiment of a hollow needle 600, wherein four conductors 602a-d are configured in an alternate pattern, different than FIG. 5. More specifically, in the embodiment of FIG. 6, conductors 602a-d are staggered. The number notation “a-d” represents all the numbers in the sequence a, b, c, and d. In this instance, each of the respective conductors may extend along and parallel with hollow needle 600. At least one conductor path 604a-d may connect from conductors 602a-d to a computing device (not illustrated) for electrical and optical signal communication. In this instance, conductor 602a may connect to the computing device via conductor path 604a. Conductor 602b may connect to the computing device via 604b, a conductor 602c may connect via a conductor path 604c, and conductor 602d may connect via conductor path 604d respectively. In some embodiments, hollow needle 600 may have an angled bevel or opening. In this instance, conductors 602a-d may appear staggered as they line the angled needle or needle bevel at the opening.

FIG. 7 illustrates a fourth embodiment of a hollow needle 700, wherein the hollow needle 700 includes inner layers that contain a conductor 702. In some embodiments, there may be an inner insulation layer 704, between the conductor and optical fiber, for keeping the electrical signal on the correct path. Hollow needle 700 may be made of metal, such as stainless steel or other materials used in the needle arts. To keep the electrical signal from grounding or following a path other than conductive path, an outer insulation layer 706, between conductor 702 and needle wall, may be present between the needle and the conductive path.

FIG. 8 is a cross sectional view of an inside of a hollow needle (not illustrated) as discussed herein. The diagram is an embodiment of a needle having four conductor resistance sensors 802a-d, although in some cases less than four, such as at least two, sensors are needed. In the embodiment of FIG. 8, four-conductor resistance sensor(s) 802a-d is illustrated showing the cross sectional view with at least four conductors 802a-d. Conductors 802a-d, may be a metal pad, or a metallization, located on or along the exterior of an inner wall of the hollow needle. A layer of insulation 806 may coat an outer wall of hollow needle. In some embodiments, a fiber 804 may be an optical fiber, which has an insulated coating or layer 806 on the outside. In some embodiments, metallization 802a-d may be in the layer of insulation 806, but in other embodiments, metallization 802a-d may be at least partially exposed to make contact with biological tissue when the hollow needle is engaged with said biological tissue (not illustrated but herein described). In the embodiment of FIG. 8, metallization 802a-d is flush with the exterior of inner wall. Thus, an object inside the lumen of hollow needle would not interfere with metallization 802a-d. One such object may include an optical fiber 804.

FIG. 9 illustrates one embodiment of an optical signal part of the system described herein (see FIG. 1). An optical fiber 904 may have a core 908 for transmitting light. Outside of core 908 may include an outer cladding 910 that reflects the light back into core 908. A hollow needle 900 may include an insulation layer 912 which insulates the electrical signal and may prevent grounding through hollow needle 900. Attached to an interior surface of insulation layer 904 may include at least one conductor 906. FIG. 9 includes four conductors, which sense electrical signals from electrical sources (not illustrated) and are sent to a computing device (not illustrated). In some examples, the outer wall of optical fiber 904 may be coated with an insulator or have a layer of insulation, such as insulation layer 912, to protect the electrical signal from grounding when the electrical signal is sent to the computing device, as described herein (see e.g. FIG. 1). In some embodiments, optical fiber 904 may include a fiber cladding 910, which may reflect light down core 908, and core 908. More specifically, at least a portion of optical fiber 904 end may be coated. The coating of the tip of optical fiber 904 may be an iron detection coating for causing a reaction when in contact with iron irons.

FIG. 10 illustrates another embodiment of the inside of a hollow needle (not illustrated). In the embodiment, two metallizations 1002 are illustrated on opposing sides of an optical fiber 1004. Metallizations may be conductors or electrodes where electricity may enter the circuit. Other arrangements or placement of each of metallizations 1002 may be possible. An insulation layer 1006 may coat the outside of optical fiber 1004, leaving each metallization 1002 at least partially exposed at the tip of a hollow needle (see, e.g., FIGS. 2 and 11) and a hollow inner portion of the hollow needle. The hollow interior may include optical fiber 1004.

FIG. 11 is an illustration of a tip of a hollow needle 1100 that is another angle of a hollow needle of FIG. 10 and is similar to FIG. 9, except with a two-conductor arrangement. In the embodiment of FIG. 11, optical fiber 1104 is seen in hollow needle 1100. Two conductors 1102 are each seen on opposing sides of fiber 1104 and on a layer of insulation 1106. Layer of insulation 1106 may encompass at least a portion of the conductors 1102. In some embodiments conductors 1102 are at least partially exposed, and conductors 1102 may be exposed so that conductors 1102 contacts the biological tissue for getting an optical signal. A conductor (or pad) can be a metallization or conductive portion that transmits electrical signal or current through its conductive material.

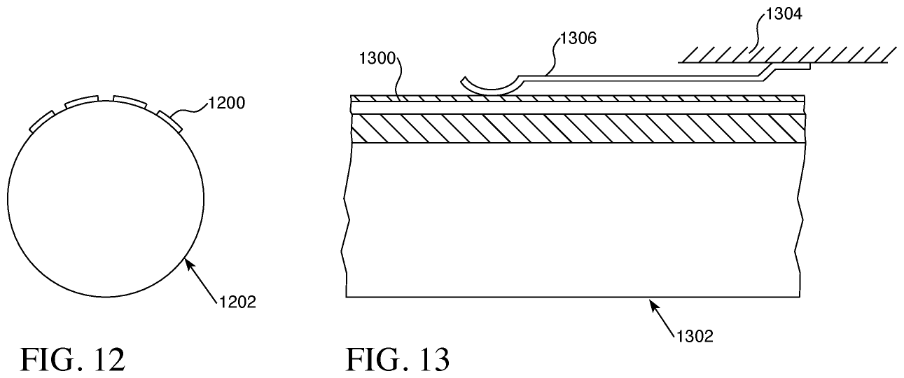

FIG. 12 illustrates an embodiment of a four-conductor resistance sensor from FIG. 8. Conductors 1200, similar to 802a-d of FIG. 8, may be located at or next to an outer wall of optical fiber 1202. In some embodiments, conductors 1200 may run along the fiber. Similar to FIGS. 5-6 and 8, there are four conductors illustrated, collectively referred to as “conductors 1200”. In FIG. 13, a conductive trace 1300 can extend along optical fiber 1302 (see, e.g., 1202). To create a circuit or data continuous data path, an arm 1306 may be in contact with one of the conductors or conductive traces 1300 to create the signal path from conductor 1200 of FIG. 12 to an electronic or computing device (see electro-optics device 118 of FIG. 1) receiving the signals and data. Arm 1306 may be attached to the inner wall of a needle 1304 or other interior surface.

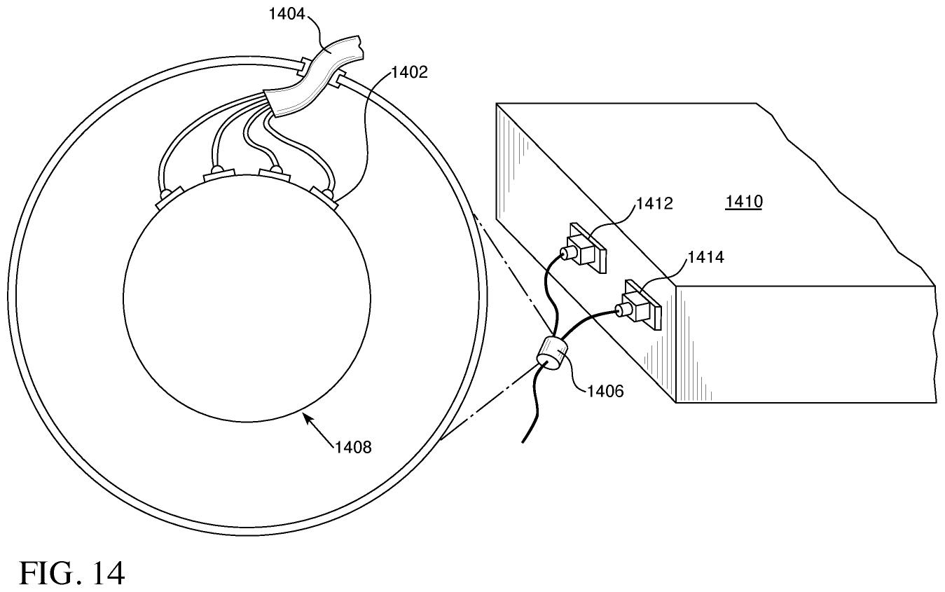

FIG. 14 is an illustration of one embodiment of the connection between the electrical and optical data to an electro-optics device 1410. An optical fiber 1408 may have at least one conductor 1402 such as the four optical fiber conductors illustrated. Conductors 1402 are each connected to electrical connections 1404. In some embodiments, the signals may transfer wirelessly, and in other embodiments, the signals may transfer via wired or optical connection, such as with an electrical interconnector 1406. The optical data from optical fiber 1408 may be received by electro-optical device 1410 via an optical fiber connector 1414 and displayed as an output for the user to see or hear. There may be a fixed electronical or optical inter-connection to fiber conductors 1404. The electrical data may be sent to and received by electro-optical device 1410 via an electrical connector 1412, and electro-optics device 1410 may use couples and processors to measure the resistance and communicate a signal or resistance value. The output may be to an LCD display, or it may be a tone, light, or other indication that the user may read or interpret.

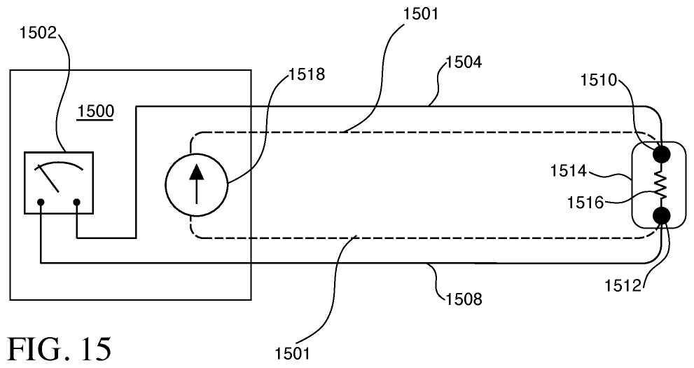

FIG. 15 illustrates an embodiment of an electrical component circuit that can calculate the resistance across the biological tissue. The resistivity of the tissue may be measured between the two electrodes on the probe tip located a significant distance away from the ohmmeter in the electro-optics module. Ohmmeter indicates R_{wire+traces}+Rₜᵢₛₛᵤₑ+R_{wire+traces}

$$R = {R}_{ref} [ 1 + α ( T - {T}_{ref} ) ]$$

Where,
R=Conductor resistance at temperature “T”
R_{ref}=Conductor resistance at reference temperature T_{ref}, usually 20° C., but sometimes 0° C.
α=Temperature coefficient of resistance for the conductor material.
T=Conductor temperature in degrees Celcius.
T_{ref}=Reference temperature that α is specified at for the conductor material.
   Different types of metals have different temperature coefficients The resistance of the wires may change with temperature. This effect can be significant as room temperature changes. The generic formula for temperature effects on resistance is as follows (at 20 degrees Celsius):
Copper=0.00393
Aluminum=0.004308
Iron=0.005866
Nickel=0.005866
Gold=0.003715
Tungsten=0.004403
Silver=0.003819

In one embodiment, two wires may be used with a constant current. Ohm's law defines resistance, “R”, as the ratio of voltage “V” across a component, to the current “I” passing through it or R=V/I. To measure resistance, a test current may be applied to a wire and the voltage drop developed may be detected. From this, the resistance may be calculated.

### Four-Wire Test Methodology With Constant Current Source

As illustrated in FIG. 15, a constant current source 1518 is provided in combination with the resistance loop (including R1_{wire+traces} 1504, R2_{wire+traces} 1508, Rₜᵢₛₛᵤₑ 1516). FIG. 15 illustrates a constant current source 1518 applied and running in a constant current loop 1501 from a first electrode 1510 and a second electrode 1512. First electrode 1510 and second electrode 1512 are placed on biomaterial or tissue 1514. Tissue 1514 has a resistance Rₜᵢₛₛᵤₑ 1516 impacting current running between first electrode 1510 and second electrode 1512. The measurement of the resistance Rₜᵢₛₛᵤₑ 1516 across the biomaterial or tissue 1514 is achieved by the electrical signal across R1_{wire+traces} 1504 to one input of a voltmeter 1502 and running in a loop across R2_{wire+traces} 1508 back to second electrode 1512. The voltmeter is within an Ohmmeter 1500 and the reading across the voltmeter is the reading between the first electrode 1510 and second electrode 1512 and that is the resistance Rₜᵢₛₛᵤₑ 1516 of tissue 1514. Ohmmeter 1500 may be contained in the electro-optic device of FIG. 1. The term “trace” or “trace impedance” may be generated from the circuit board components and the impede due to materials used.

In general, a 4-wire measurement may eliminate any effect of fixture resistance (the lead wires) to obtain a precise resistance value of the tissue resistance. Different tissues have different resistivity, and the following are some examples of biological tissue and the resistivity:

| Tissues | S/m |
|---|---|
| Cerebellum | 0.10 |
| C.S.F. | 2.00 |
| Cornea | 0.40 |
| Eye humor | 1.50 |
| Grey matter | 0.10 |
| Hypothalamus | 0.08 |
| Eye lens | 0.25 |
| Pineal body | 0.08 |
| Pituitary | 0.08 |
| Salivary gland | 0.35 |
| Thalamus | 0.08 |
| Tongue | 0.30 |
| White matter | 0.06 |
| Adrenals | 0.35 |
| Bladder | 0.20 |
| Large intestine | 0.10 |
| Duodenum | 0.50 |
| Esophagus | 0.50 |
| Bile | 1.40 |
| Gall bladder | 0.20 |
| Heart | 0.10 |
| Kidney | 0.10 |
| Liver | 0.07 |
| Lung | 0.14 |
| Large intestine | |
| Contents | 0.35 |
| pancreas | 0.35 |
| prostate | 0.40 |
| small intestine | 0.50 |
| spleen | 0.10 |
| stomach | 0.50 |
| stomach contents | 0.35 |
| tendon | 0.30 |
| testis | 0.35 |
| thyroid gland | 0.50 |
| trachea | 0.35 |
| urine | 0.70 |
| blood | 0.70 |
| cortical bone | 0.02 |
| bone marrow | 0.06 |
| cartilage | 0.18 |
| fat | 0.04 |
| muscle | 0.35 |
| nerve (spinal cord) | 0.03 |
| skin | 0.10 |
| tooth | 0.02 |
| ligament | 0.30 |
| diaphragm | 0.35 |
| seminal vesicle | 0.35 |
| cavernous body | 0.35 |
| small intestine | |
| contents | 0.35 |

In the present invention, reading accuracy of Rₜᵢₛₛᵤₑ, may be improved by moving the voltage measurement points out to the endpoints of the mating pins. Thus, bypassing any voltage drop that may occur in the lead wires. Because some embodiments use four lead wires instead of two, this approach is referred to as “4-wire measurement,” or alternatively “4-Wire Kelvin.”

In some embodiments, volt meter 1502 has very high impedance so the current flowing through the voltage-measuring circuit of a 4-wire system is extremely small, typically on the order of fractions of a microamp, so virtually no voltage drop occurs across these lead wires, and the effect on the resistance measurement is negligible. In summary, if there is no current flowing through a wire, there may be no voltage drop across it. In one embodiment, a voltmeter may be an analog-to-digital convertor chip.

### Two-Wire Test Methodology With Constant Voltage Source

In another embodiment, a constant voltage may be applied to the detection electrodes and the current drawn would be measured. In this embodiment, the circuit is similar to FIG. 15, but constant current loop 1518 may not be present. Ohm's law defines resistance, “R”, as the ratio of voltage “V” across a component, to the current “I” passing through it: R=V_{ref}/I. So the current draw will be: I=V_{ref}/R. The resistance is then equal to: R=Vref/I.

### Four-Terminal Bulk Resistivity Measurement

In yet another embodiment, a four-probe method may be used that involves using four equally-spaced, co-linear probes (known as a four-point probe) to make electrical contact with the material. Other configurations are also suitable depending on geometrical space constraints. This electrical impedance measuring technique uses separate pairs of current-carrying and voltage-sensing electrodes to make more accurate measurements than the simpler and more usual two-terminal sensing. The current may be run between the two outer probes while the voltage sensing is done on the inner probes.

Separation of current and voltage electrodes eliminates the lead and contact resistance from the measurement. This is an advantage for precise measurement of low resistance values. Alternating current can also be used to measure the impedance of the tissue in the sensor. Furthermore, testing at different frequencies can provide even more information about the tissue under test.

Resistance R0 is the desired tissue resistivity measurement, R1 & R2 are the current loop contact resistances, R3 & R4 are the voltage loop contact resistances, R5 & R6 are the resistances between current and voltage loop contacts. Different configurations are possible for the resistance.

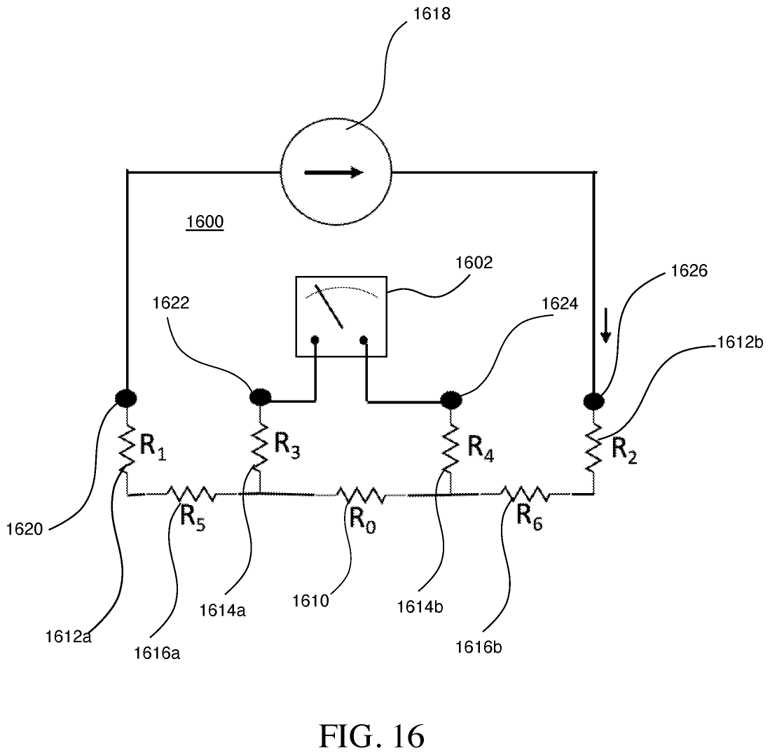

FIG. 16 illustrates a typical technique used for measuring bulk resistance in a four-probe method. In such a technique, there may be four equally-spaced, co-linear probes (known as a four-point probe) to make electrical contact with a material. Other embodiments may include additional configurations depending on geometrical space constraints. FIG. 16 illustrates a constant current source 1618 applied and running in a loop from a first electrode 1620, a second electrode 1622, a third electrode 1624, and a fourth electrode 1626. The voltmeter 1602 is within an Ohmmeter 1600. Such electrical impedance measuring techniques can use separate pairs of current-carrying and voltage-sensing electrodes to make more accurate measurements than a simpler two-terminal sensing technique. In a preferred embodiment can include a current that may run between the two outer probes while the voltage sensing is done on the two inner probes. The separation of current and voltage electrodes can eliminate the lead and contact resistance from a resistance measurement. This can be advantageous for the precise measurement of low resistance values. Resistance R0 1610 is the desired tissue resistivity measurement, R1 & R2 are the current loop contact resistances 1612a and 1612b; respectively. R3 & R4 are the voltage loop contact resistances 1614a and 1614b; respectively. R5 & R6 are the resistances between current and voltage loop contacts 1616a and 1616b; respectively.

Persons of ordinary skill in arts relevant to this disclosure and subject matter hereof will recognize that embodiments may comprise fewer features than illustrated in any individual embodiment described by example or otherwise contemplated herein. Embodiments described herein are not meant to be an exhaustive presentation of ways in which various features may be combined and/or arranged. Accordingly, the embodiments are not mutually exclusive combinations of features; rather, embodiments can comprise a combination of different individual features selected from different individual embodiments, as understood by persons of ordinary skill in the relevant arts. Moreover, elements described with respect to one embodiment can be implemented in other embodiments even when not described in such embodiments unless otherwise noted. Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless stated that a specific combination is not intended. Furthermore, it is also intended to include features of a claim in any other independent claim, even if this claim is not directly made dependent on the independent claim.

1-20. (canceled)

21. A real-time impedance measuring needle system comprising:
a needle having a distal end, a proximal end, and a fluid channel disposed therethrough, wherein the proximal end and the distal end each include an opening;
at least two conductors disposed near the opening of the distal end of the needle; and
at least one detector configured to sense an electrical current running through a biological tissue disposed between the at least two conductors.

22. The real-time impedance measuring needle system according to claim 21, wherein an impedance measurement is derived from the electrical current of the biological tissue disposed between the at least two conductors.

23. The real-time impedance measuring needle system according to claim 21, wherein the electrical current between the at least two conductors are one of constant current or alternating current.

24. The real-time impedance measuring needle system according to claim 21, wherein an indication of a location of the needle is an output of the detector.

25. The real-time impedance measuring needle system according to claim 24, wherein the output varies on a resistivity of the biological tissue.

26. The real-time impedance measuring needle system according to claim 25, wherein the output further comprises a numerical representation based on the resistance of the biological tissue.

27. The real-time impedance measuring needle system according to claim 25, wherein a diameter of the fluid channel is configured to receive an optical fiber.

28. The real-time impedance measuring needle system according to claim 27, wherein the at least two conductors are on opposing sides of the needle and the optical fiber is disposed within the fluid channel.

29. The real-time impedance measuring needle system according to claim 28, wherein the optical fiber further comprises an iron detection coating.

30. The real-time impedance measuring needle system according to claim 21, further comprising the needle having an inner wall, wherein the at least two conductors are insulated from the inner wall and attached thereto or integrated therein.

31. The real-time impedance measuring needle system according to claim 30, wherein the at least two conductors extend along the inner wall of the needle forming a conducting path from the distal end to the proximal end, and further wherein the conducting path connects to a computing device.

32. The real-time impedance measuring needle system according to claim 21, wherein the needle has an inner conductor and an outer conductor.

33. The real-time impedance measuring needle system according to claim 21, wherein the detector contains at least one processor and a coupler.

34. The real-time impedance measuring needle system according to claim 21, wherein the hollow needle has a flange for contacting the conductor.

35. The real-time impedance measuring needle system according to claim 27, wherein a second output is based on an optical signal received by the detector via the optical fiber.

36. The real-time impedance measuring needle system according to claim 21, wherein the at least one detector comprises a circuit board that includes one or more wireless interfaces.

37. A real-time impedance measuring needle system comprising:
a needle having a bore, a proximal end, and a lumen disposed therethrough;
at least two conductors disposed at the bore; and
at least one detector, wherein each detector includes at least one processor, and further wherein the at least one detector is configured to sense an electrical current within a biological tissue disposed between the at least two conductors.

38. The real-time impedance measuring needle system of claim 37, further comprising a detector with a coupler.

39. A method of measuring tissue impedance in real-time comprising the steps of:
providing a needle having a distal end, a proximal end, and a fluid channel disposed therethrough, wherein the proximal end and the distal end each include an opening;
at least two conductors disposed near the opening of the distal end of the needle; and
at least one detector configured to sense an electrical current running through a biological tissue disposed between the at least two conductors;
inserting the needle into the biological tissue;
sensing with the at least one detector an electrical current running through the biological tissue disposed between the at least two conductors; and
generating an output based on the electrical current.

40. The method of measuring tissue impedance in real-time of claim 39, comprising the further step of repositioning the needle in response to the output until a desired output is generated.
